(19) United States
(12) Patent Application Publication
(10) Pub. No.: US 2026/0061226 A1
(43) Pub. Date: Mar. 5, 2026

(54) Transducer Probe for Direct Ultrasonic Neuromodulation and Measurement

(71) Applicant: Alien Sandbox, LLC, Austin, TX (US)

(72) Inventors: Neal A. Hall, Austin, TX (US); Zihuan Liu, Austin, TX (US); Ehsan Vatankhah, Austin, TX (US); Yuqi Meng, Austin, TX (US); Xiaoyu Niu, Austin, TX (US)

(21) Appl. No.: 19/317,723

(22) Filed: Sep. 3, 2025

Related U.S. Application Data
(60) Provisional application No. 63/690,076, filed on Sep. 3, 2024.

(51) Int. Cl. A61N 7/00

(52) U.S. Cl. CPC A61N 7/00; A61N 2007/0026; A61N 2007/0043; A61N 2007/0078; A61N 2007/0095

(57) ABSTRACT

Systems, methods, and mechanisms for a transducer probe for ultrasonic measurement and direct modulation, e.g., a transducer probe generating ultrasound across a broad range of frequencies to acoustically modulate groups of neurons. An ultrasonic probe can include an array of ultrasonic transducers, a substrate, and a tube. The array of ultrasonic transducers can be included on the substrate and the substrate can be positioned and/or included within an interior of the tube. In addition, a volume between the substrate and an interior wall of the tube can be filled with an acoustic substance configured to provide acoustic coupling between the substrate and the tube. The ultrasonic probe can be configured to radiate, via the array of ultrasonic transducers positioned within the acoustic medium, ultrasound through the acoustic medium.

### PRIORITY DATA

This application claims benefit of priority to U.S. Application No. 63/690,076, titled “Transducer Probe for Direct Ultrasonic Neuromodulation and Measurement”, filed Sep. 3, 2024, which is hereby incorporated by reference in its entirety as though fully and completely set forth herein.

### FIELD OF THE INVENTION

This disclosure generally relates to systems, methods, and mechanisms for a transducer probe for ultrasonic measurement and direct neuromodulation, e.g., a transducer probe generating ultrasound across a broad range of frequencies to acoustically modulate groups of neurons.

### DESCRIPTION OF THE RELATED ART

Nearly 100 million Americans are affected by psychiatric and neurological disorders with a significant burden to society costing more than $765 billion dollars. Many of these disorders have limited treatment options and disease remission is not always achieved. Thus, there is a critical need for new innovative methods and technologies for precise neuromodulation of discrete brain circuits involved in these diseases.

In existing implementations, electrical deep brain stimulation (DBS) is an established clinical technique in which a probe, approximately 1 mm in diameter, is embedded into a brain. Electrical signals, generated via the probe, create an electric field in the brain that modulates neural activity. The electrical signals, or electrical impulses, may regulate abnormal impulses in the brain and/or may affect certain cells and chemicals within the brain. Stimulation level is often controlled via a controller (e.g., a pacemaker-like device) implanted under a patient's skin in the upper left chest. A wire, also embedded in the patient, travels from the controller to the probe (or probes). DBS is commonly used to treat numerous conditions such as Parkinson's disease, essential tremor, dystonia, epilepsy, and obsessive-compulsive disorder. In addition, DBS is being studied as a potential treatment for Tourette syndrome, Huntington's disease and chorea, chronic pain, and cluster headache.

The probe may be made up of multiple electrodes that allow for different electric field geometries to be generated in the brain. The various electric field geometries may be controlled to target various regions of the brain for stimulation. Signals generated by the probe may be static voltages (e.g., direct current (DC) voltages) or dynamic voltages (e.g., alternating current (AC) voltages). However, focusing, control, and/or steering of the electric field is limited. Therefore, further improvements in the field are desired.

### SUMMARY OF THE INVENTION

Various embodiments of systems, methods, and mechanisms for a transducer probe for ultrasonic measurement and direct modulation, e.g., a transducer probe generating ultrasound across a broad range of frequencies to acoustically modulate groups of neurons, are described herein.

For example, in some embodiments, an ultrasonic probe can include an array of ultrasonic transducers, a substrate, and a tube. The array of ultrasonic transducers can be included on the substrate and the substrate can be positioned and/or included within an interior of the tube. In addition, a volume between the substrate and an interior wall of the tube can be filled with an acoustic substance configured to provide acoustic coupling between the substrate and the tube. The acoustic substance can be a gel and/or an acoustically transparent material. The gel can also be and/or be considered an acoustically transparent material. The ultrasonic probe can be configured for intra-acoustic medium ultrasonic modulation via insertion into an acoustic medium. As such, the ultrasonic probe can be configured to radiate, via the array of ultrasonic transducers positioned within the acoustic medium, ultrasound through the acoustic medium.

As another example, in some embodiments, ultrasound can be radiated through an acoustic medium, e.g., via an array of ultrasonic transducers positioned within the acoustic medium. In some instances, the array of ultrasonic transducers can be comprised on a substrate that resides in an interior of a tube to form an ultrasonic probe. Additionally, a volume between the substrate and an interior wall of the tube can be filled with an acoustic substance (e.g., an acoustically transparent material, such as a gel) configured to provide acoustic coupling between the substrate and the tube. In addition, neural activity can be monitored responsive to the radiated ultrasound and a location and/or size of a focal spot or region of the radiated ultrasound can be steered, e.g., based on the monitoring. Further, a treatment zone, e.g., a group and/or volume of neurons, within the acoustic medium can be identified, e.g., based on the steering. In other words, as the location and/or size of the focal spot or region of the radiated ultrasound is steered through the acoustic medium, patient behavior/response can be monitored (e.g., via sensed neural activity and/or observation) to determine and/or identify the group and/or volume of neurons for treatment.

This Summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The following detailed description makes reference to the accompanying drawings, which are now briefly described.

FIGS. 1A and 1B illustrate a possible example of a needle-style ultrasonic transducer probe, according to some embodiments.

FIG. 2 illustrates another possible example of a probe, according to some embodiments.

FIGS. 3A, 3B, and 3C illustrate further possible examples of a probe, according to some embodiments.

FIGS. 4A-4E illustrate another example of a probe, e.g., a probe, according to some embodiments.

FIGS. 5, 6, and 7 illustrate block diagrams of examples of methods for intra-acoustic medium ultrasonic neuromodulation, according to some embodiments.

While the invention is susceptible to various modifications and alternative forms, specific embodiments thereof are shown by way of example in the drawings and are herein described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

### Terms

The following is a glossary of terms used in this disclosure:

Memory Medium—Any of various types of non-transitory memory devices or storage devices. The term “memory medium” is intended to include an installation medium, e.g., a CD-ROM, floppy disks, or tape device; a computer system memory or random-access memory such as DRAM, DDR RAM, SRAM, EDO RAM, Rambus RAM, etc.; a non-volatile memory such as a Flash, magnetic media, e.g., a hard drive, or optical storage; registers, or other similar types of memory elements, etc. The memory medium may include other types of non-transitory memory as well or combinations thereof. In addition, the memory medium may be located in a first computer system in which the programs are executed or may be located in a second different computer system which connects to the first computer system over a network, such as the Internet. In the latter instance, the second computer system may provide program instructions to the first computer for execution. The term “memory medium” may include two or more memory mediums which may reside in different locations, e.g., in different computer systems that are connected over a network. The memory medium may store program instructions (e.g., embodied as computer programs) that may be executed by one or more processors.

Carrier Medium—a memory medium as described above, as well as a physical transmission medium, such as a bus, network, and/or other physical transmission medium that conveys signals such as electrical, electromagnetic, or digital signals.

Functional Unit (or Processing Element/Processor)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors, as well as any combinations thereof.

Processing Element/Processor (or Functional Unit)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors.

Programmable Hardware Element—includes various hardware devices comprising multiple programmable function blocks connected via a programmable interconnect. Examples include FPGAs (Field Programmable Gate Arrays), PLDs (Programmable Logic Devices), FPOAs (Field Programmable Object Arrays), and CPLDs (Complex PLDs). The programmable function blocks may range from fine grained (combinatorial logic or look up tables) to coarse grained (arithmetic logic units or processor cores). A programmable hardware element may also be referred to as “reconfigurable logic”.

Computer System (or Computer)—any of various types of computing or processing systems, including a personal computer system (PC), mainframe computer system, workstation, network appliance, Internet appliance, personal digital assistant (PDA), television system, grid computing system, or other device or combinations of devices. In general, the term “computer system” can be broadly defined to encompass any device (or combination of devices) having at least one processor that executes instructions from a memory medium.

Sound/Soundwave—refers to mechanical and/or elastic waves (including surface acoustic waves) in any medium (gas, liquid, solid, gel, soil, and so forth) and/or combination of media, including interfaces between media. Sound may occur at any amplitude and at any frequency, e.g., ranging from infrasound (e.g., sound waves with frequencies below human audibility, generally considered to be under 20 hertz (Hz)) to ultrasound (e.g., sound waves with frequencies above human audibility, generally considered to be above 20 kilohertz (kHz)), as well as all frequencies in between.

Sound Pressure (or Acoustic Pressure)—refers to a local pressure and/or stress deviation from an ambient (quiescent, average, and/or equilibrium) pressure, caused by a sound wave.

Sound Beam—refers to an area through which sound energy emitted from a sound transducer travels. A sound beam may be three dimensional and symmetrical around its central axis. In some instances, e.g., depending on a sound transducer implemented to generate the sound beam, a sound beam may include a converging region and a diverging region. The regions may intersect at a focal point of the sound beam. In other instances, e.g., depending on a sound transducer implemented to generate the sound beam, a sound beam may include a near field (or Fresnel zone) which is cylindrical in shape and a far field (or Fraunhofer zone) which is conical in shape and in which the sound beam diverges. A sound beam may be a continuous wave, single-frequency beam. Additionally, a sound beam may be tone-bursts, e.g., comprised of a finite number of cycles at a particular frequency. Further, a sound beam may be broadband, comprised of a finite duration pulse and having a broad spectral content.

Medium (or Acoustic Medium)—refers to any substance through which sound may pass, including, but not limited to a gas, a liquid, a solid, a gel, soil, and so forth. Example of mediums may include, but are not limited to, various portions of human anatomy, including soft tissue such as muscle (e.g., heart) and organs (e.g., brain, kidney, lungs, and so forth) as well as hard tissue (e.g., such as bones), as well as various layers of the earth, including the crust, the upper mantle, the mantle, the outer core, and the inner core.

Automatically—refers to an action or operation performed by a computer system (e.g., software executed by the computer system) or device (e.g., circuitry, programmable hardware elements, ASICs, etc.), without user input directly specifying or performing the action or operation. Thus, the term “automatically” is in contrast to an operation being manually performed or specified by the user, where the user provides input to directly perform the operation. An automatic procedure may be initiated by input provided by the user, but the subsequent actions that are performed “automatically” are not specified by the user, i.e., are not performed “manually”, where the user specifies each action to perform. For example, a user filling out an electronic form by selecting each field and providing input specifying information (e.g., by typing information, selecting check boxes, radio selections, etc.) is filling out the form manually, even though the computer system must update the form in response to the user actions. The form may be automatically filled out by the computer system where the computer system (e.g., software executing on the computer system) analyzes the fields of the form and fills in the form without any user input specifying the answers to the fields. As indicated above, the user may invoke the automatic filling of the form but is not involved in the actual filling of the form (e.g., the user is not manually specifying answers to fields but rather they are being automatically completed). The present specification provides various examples of operations being automatically performed in response to actions the user has taken.

Approximately/Substantially—refers to a value that is almost correct or exact. For example, approximately may refer to a value that is within 1 to 10 percent of the exact (or desired) value. It should be noted, however, that the actual threshold value (or tolerance) may be application dependent. For example, in one embodiment, “approximately” may mean within 0.1% of some specified or desired value, while in various other embodiments, the threshold may be, for example, 2%, 3%, 5%, and so forth, as desired or as required by the particular application. Furthermore, the term approximately may be used interchangeably with the term substantially. In other words, the terms approximately and substantially are used synonymously to refer to a value, or shape, which is almost correct or exact.

Couple—refers to the combining of two or more elements or parts. The term “couple” is intended to denote the linking of part A to part B, however, the term “couple” does not exclude the use of intervening parts between part A and part B to achieve the coupling of part A to part B. For example, the phrase “part A may be coupled to part B” means that part A and part B may be linked indirectly, e.g., via part C. Thus, part A may be connected to part C and part C may be connected to part B to achieve the coupling of part A to part B.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third component electrically connected to the module substrate” does not preclude scenarios in which a “fourth component electrically connected to the module substrate” is connected prior to the third component, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 (f) interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### Transducer Probe for Ultrasonic Measurement and Neuromodulation

In current implementations, electrical deep brain stimulation (DBS) is an established clinical technique in which a probe, approximately 1 mm in diameter, is embedded into a brain. Electrical signals, generated via the probe, create an electric field in the brain that modulates neural activity. The electrical signals, or electrical impulses, may regulate abnormal impulses in the brain and/or may affect certain cells and chemicals within the brain. Stimulation level is often controlled via a controller (e.g., a pacemaker-like device) implanted under a patient's skin in the upper left chest. DBS is commonly used to treat numerous conditions such as Parkinson's disease, essential tremor, dystonia, epilepsy, and obsessive-compulsive disorder. In addition, DBS is being studied as a potential treatment for Tourette syndrome, Huntington's disease and chorea, chronic pain, and cluster headache. The probe may be made up of multiple electrodes that allow for different electric field geometries to be generated in the brain. The various electric field geometries may be controlled to target various regions of the brain for stimulation. Signals generated by the probe may be static voltages (e.g., direct current (DC) voltages) or dynamic voltages (e.g., alternating current (AC) voltages). However, control, focusing, and/or steering of the electric field is limited.

Low-intensity focused ultrasound (LIFU) is another novel neuromodulatory approach that uses mechanical energy to modulate neuronal activity with high spatial resolution and adjustable depth of focus. LIFU has been used safely and effectively for neuromodulation in various ranges of animals, including mice, rats, rabbits, sheep, pigs, and primates. LIFU has also been an effective method for transient cortical and sub-cortical neuromodulation in humans. However, despite its enormous promise, there are considerable gaps that preclude LIFU from immediate translation to human clinical treatment options. Critically, the effect of LIFU on individual neurons in humans is unknown. LIFU modulation may have varying effects depending on neuronal type and activity, brain region, and also the particular waveform parameters applied. Thus, there is a need to understand how ultrasound interacts with neurons in the human brain so that the technology can be deployed rationally and with predictable clinical effects.

There has been some success with single unit activity in response to LIFU in awake humans during movement disorder surgeries where implanted electrodes are able to simultaneously record the responses from single neurons and their local field potentials (LFPs). That study found that 5 minutes of pulsed LIFU changed sub-thalamic nucleus (STN) LFP activity from a baseline of approximately 20 Hz to approximately 30 Hz that persisted upon cessation of LIFU. This finding is similar to research exploring how dopaminergic agents affect STN firing in Parkinson's disease. Despite these significant results, currently LIFU is delivered through the skull using a 500 kHz single element that precludes sub-millimeter resolution and precise control of beam magnitude and geometry—as the skull is the primary barrier to ultrasound. Therefore, improvements are desired.

Embodiments described herein provide systems, methods, and mechanisms for a transducer probe for ultrasonic measurement and direct neuromodulation, e.g., a transducer probe generating ultrasound across a broad range of frequencies to acoustically modulate groups of neurons. Further, embodiments described herein address various issues related to ultrasonic neuromodulation, including, but not limited to, developing the relationship between neuron behavior and ultrasound, e.g., via enabling a mapping of ultrasonic frequency, ultrasonic intensity, neuron type, and brain region to behavior of individual neurons or small groups of neurons. For example, the ability to stimulate and record in vivo neuronal activity in response to LIFU may provide a fundamental understanding of LIFU neuromodulation from the single neuron to brain circuit and ultimately to behavior. Such information may lay a foundation to use ultrasound diagnostically for brain mapping and therapeutically for targeted psychiatric and neurological interventions and treatments. Benefits of in vivo measurements may include, but are not limited to, direct, real-time recording of neural spiking activity, high sensitivity to measure single (and groups of) neurons activity and response, exquisite spatial and temporal resolution, high signal to noise ratio, no confound of anesthesia (human studies), no confound of skull, and no scalability or translational issues.

Note that the embodiments described herein may aid in advancement of a fundamental scientific understanding of acoustic neuromodulation, e.g., may aid experiments aimed at understanding how and/or why ultrasound modulates neurological activity and understanding the mapping of input parameters (vibration amplitude, frequency, target region and size) to various neurological output parameters. In addition, embodiments described herein may replace and/or advance electrical DBS in humans at least because ultrasonic DBS could overcome issues related to focusing, controlling, and steering of an electric field that occur with electrical DBS.

In some embodiments, sound may be focused using phased array imaging. A focal spot or a focal region may be moved about by controlling input signals using phased array techniques as known to those skilled in the art. In some instances, the focal region may be positioned in close proximity to the probe (e.g., 1 mm from probe axis) or a distance away from the probe (e.g., 2 or 3 cm away). In some instances, the focal region may be steered, e.g., an ultrasound beam emitted from the array can be steered left to right. Additionally, in some instances, multiple focal regions can be generated simultaneously, e.g., selection of driving signals to each ultrasonic transducer can be such that two or three distinct focal regions are generated at the same time and, at least in some instances, in different directions. These multiple focal regions may be moved about in time to achieve a so-called spatiotemporal neuromodulation.

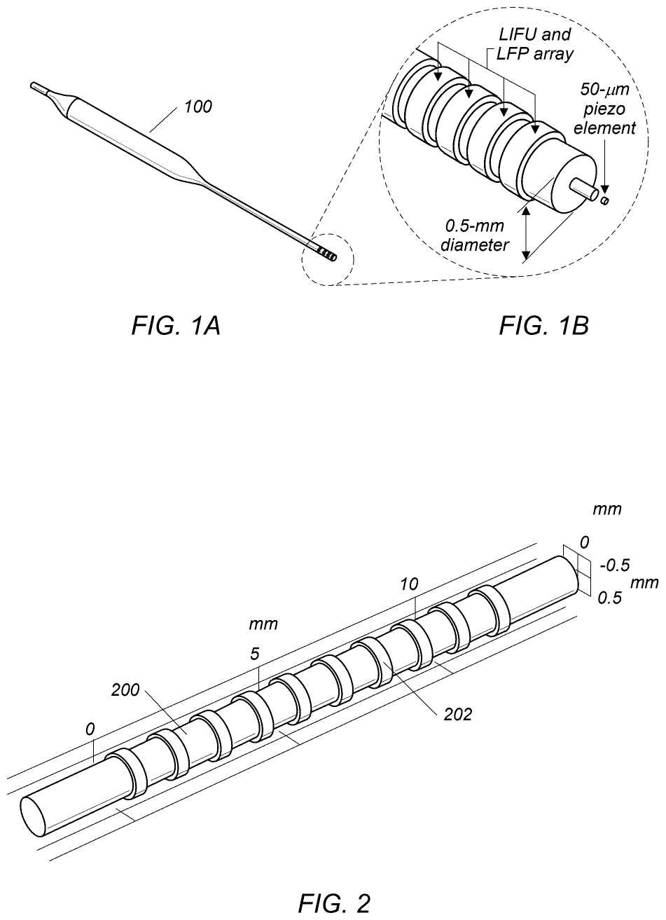

In some embodiments, a needle-style ultrasonic transducer probe may be configured to generate focused ultrasound inside living brains. When inserted in brains of living animals and humans, the probe may generate focused ultrasound in any direction and across a frequency range 0.25 megahertz (MHz) to 5 MHz. In addition, the probe may be configured such that the ultrasound may be focused to a 100 μm diameter spot. In some embodiments, the ultrasound may be focused in the immediate vicinity of the probe, e.g., within 10 percent of a longitudinal length, L, of the probe (e.g., 0.1L) or as far out as 2 times the longitudinal length of the probe (e.g., 2L). In some instances, the phased array may be integrated with electrodes configured to measure local field potentials (LFPs). LFPs recordings may be subsequently filtered in frequency to understand which LFP signal components result from synaptic firings of relatively large groups of neurons versus which LFP signal components are due to action potentials of neurons in immediate vicinity of the probe. FIGS. 1A and 1B illustrate a possible example of a needle-style ultrasonic transducer probe, according to some embodiments. Note that needle-style is not meant to imply rigidity. Needle-style is meant to imply a slender aspect ratio. Thus, a needle-style probe may be approximately (or substantially) rigid, nominally rigid, nominally flexible, and/or flexible. As shown, the needle-style ultrasonic transducer probe 100 may include both a low-intensity focused ultrasound (LIFU) array and an LFP array. In addition, in at least some instances, the needle-style ultrasonic transducer probe 100 may further include an ultra-small piezo electric element at its tip that may be configured to stimulate a single cell. As shown, the piezo electric element may be approximately 50 μm, e.g., approximately one-tenth the diameter of the needle-style ultrasonic transducer probe 100.

In some embodiments, the needle-style ultrasonic transducer probe 100 (e.g., probe) may be configured to direct ultrasonic dosing of a single or small group of neurons in living brains and simultaneously perform electrical measurement of the response of those neurons. The probe may be configured to enter a living brain (human or animal) much like an electrical deep brain stimulation (DBS) probe with an exact location of the probe in relation to internal regions of the brain precisely known using X-ray or magnetic resonance imaging (MRI) taken prior to ultrasonic experimentation. Once in the target position, the probe may be configured to generate a focus ultrasound with a high spatially resolved focal point (e.g., approximately 100 μm focal diameter) to excite a small group of neurons. In some instances, for an even higher spatial resolution, an individual transducer element with a 50 μm diameter may be integrated onto the tip of the probe as shown in FIG. 1B. Such a transducer, with a diameter on the order of a single neuron, may vibrate or transmit in either direct contact with or in immediate proximity of a targeted region. As such, direct and highly controllable dosing of a single neuron or a small number of neurons may be possible over a broad and controllable range of frequencies (e.g., such as DC to 10 MHz) and vibration amplitudes.

In addition, the probe illustrated in FIGS. 1A and 1B may also include micro electrodes for measurement of local field potential (LFP) waveforms. Note that current understanding of LFP signals is that LFPs may result from a combination of action potentials from neurons in direct contact with the electrode and from synchronized synaptic currents in larger groups of neurons not in immediate contact with the electrode. The former may be distinguishable from the latter using frequency-selective filtering. Additionally, action potentials are known to generate higher frequency spiking as compared to lower frequency waves (below 100 Hz) generated by synaptic currents.

Thus, in at least some embodiments, the probe may combine multiple capabilities, e.g., (1) a focused ultrasound spot with 100 μm diameter may be steered to any point in proximity of the probe (e.g., within 10L of the probe and (2) electrodes on the probe may measure LFPs resulting from the neuromodulation. Note that an addressable acoustic field may include points in direct contact with the LFP electrodes, e.g., ultrasound may be focused onto a single neuron and LFPs which comprise action potentials from that same neuron may be directly measured.

In some embodiments, the probe may be combined with traditional electrical DBS. For example, the probe may include an ultrasonic transducer array and electrical DBS electrodes that are used to generate an electrical field. Further, in some instances, the probe may also include neuro sensors, such as local field potential sensors. Such a probe may thus include multiple types of neuromodulation (ultrasound and electrical) with integrated neuro sensors. In addition, electrodes along the probe may share various functions. For example, an electrode configured for ultrasonic actuation at an ultrasonic frequency may also be configured to apply an electric field at the same or a different frequency or at DC. In some cases, the two types of neuromodulation may be applied simultaneously, using the same shared electrode. In other words, the shared electrode may be configured for ultrasonic actuation and electric field actuation.

As noted above, in some embodiments, the probe may include a plurality of ultrasonic transducer elements, each ultrasonic transducer element individually controllable. Thus, in some embodiments, a focal point of the ultrasound may be configurable (e.g., in both terms of size of the focal point and location of the focal point) to acoustically modulate a small group of neurons, e.g., a group of neurons within an area/volume defined by a 100-micrometer diameter spherical region.

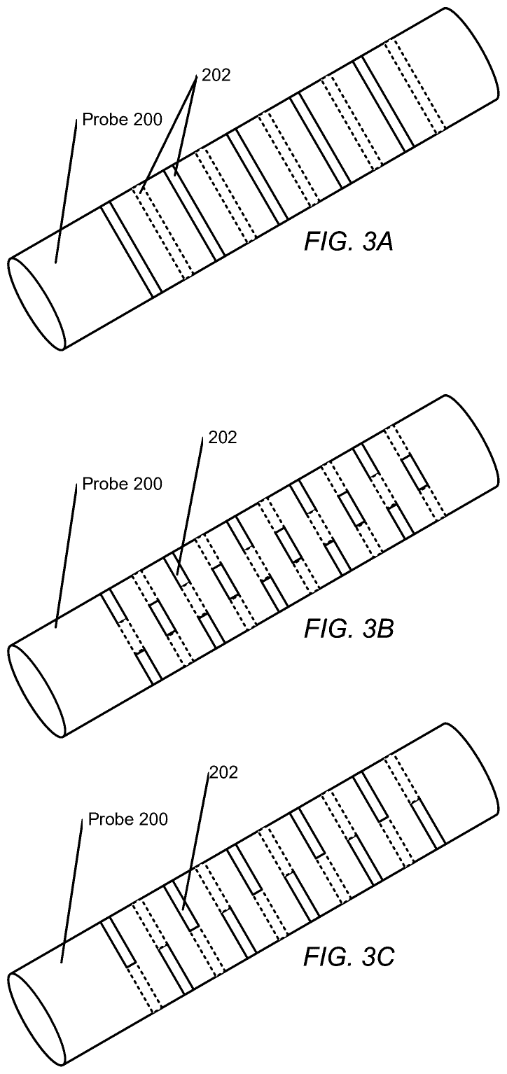

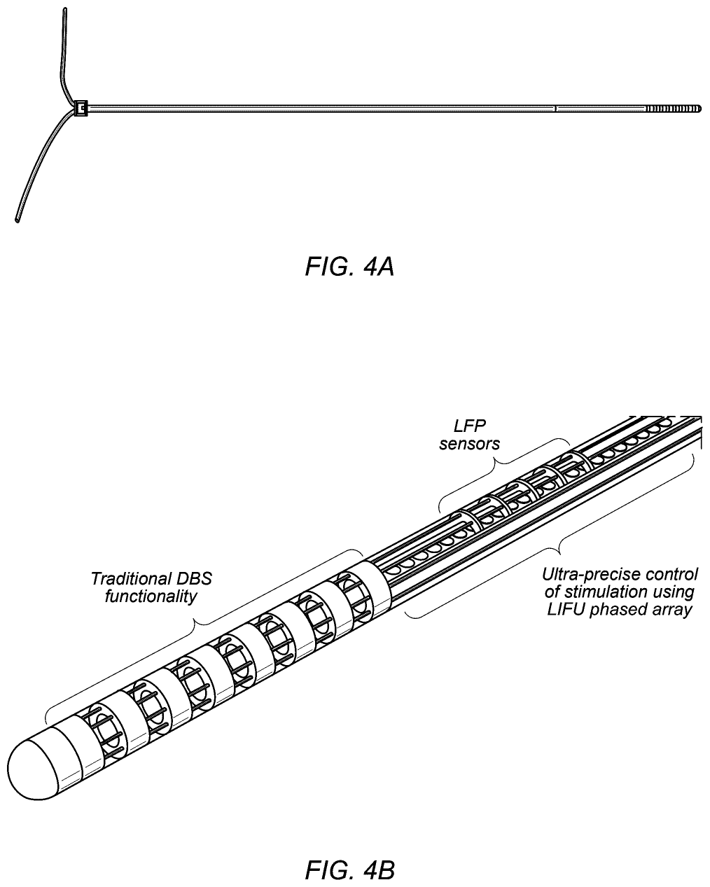

As illustrated by FIG. 2, a probe 200, which may be an example of the needle-style ultrasonic transducer probe 100, for example, may have a diameter of approximately 0.5 millimeters (mm) and include a plurality of ultrasonic transducer elements 202 along its length. For example, the probe 200 may include 10 or more ultrasonic transducer elements 202 along its length. As shown in FIG. 2, the plurality of ultrasonic transducer elements 202 may be arranged along the length of the probe 200. Additionally, as shown in FIGS. 3A, 3B, and 3C, the plurality of ultrasonic transducer elements 202 may also be arranged along the circumference of the probe. For example, as shown in FIG. 3A, the ultrasonic transducer elements 202 may be “split” such that each transducer encompasses half the circumference of the probe 200. Thus, as shown in FIG. 3A, the ultrasonic transducer elements 202 may alternate sides of the probe 200. Note that other geometries are possible as well, e.g., as illustrated by FIG. 3B (ultrasonic transducer elements 202 are “split” such that each transducer element 202 encompasses one-third the circumference of the probe 200) and FIG. 3C (ultrasonic transducer elements are “split” such that each transducer element 202 encompasses one-fourth the circumference of the probe 200). Note that independent of the geometric configuration, each of the plurality of ultrasonic transducer elements 202 may be individually addressable (e.g., each of the plurality of ultrasonic transducer elements may be independently controlled to produce soundwaves) and the probe 200 may be configured to generate ultrasound across a broad range of frequencies.

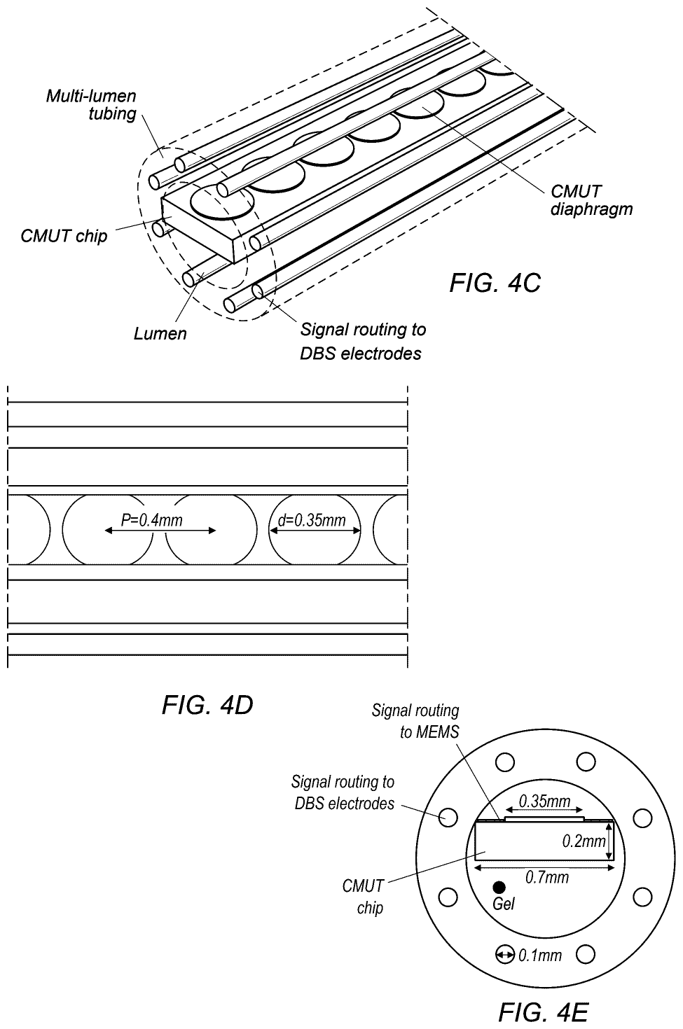

FIGS. 4A-4E illustrate another example of a probe, e.g., a probe, according to some embodiments. As shown in FIGS. 4A-4E, a probe 400, which may be an example of the needle style ultrasonic transducer probe 100, can include traditional and/or legacy deep brain stimulation (DBS) functionality (e.g., probe 400 can include electrical stimulation elements as used in traditional DBS, e.g., electrical DBS electrodes) as well as LFP sensors forming an LIFU phased array configured for ultra-precise control of sound based stimulation. In some instances, the probe can be considered an ultrasonic DBS (UDBS) probe. Further, probe 400 can be formed using tubing which has a hollow interior. In some instances, the tubing can be tubing. The tubing can have multiple lumens or conduits that can route signals down a length of probe 400 to the electrical DBS electrodes and to neuro-sensing electrodes. The hollow interior of the tubing can house a MEMS chip with a linear array of ultrasonic transducer elements (e.g., capacitive micromachined ultrasonic transducers (CMUTs), as shown, piezoelectric micromachined ultrasonic transducers (PMUTs), and/or 3-D printed piezoelectric elements). As one example, the linear array of ultrasonic transducers can include a 24-element array spanning a distance of approximately 1 centimeter along the interior of the tubing. In some instances, a remaining space in the tube interior (e.g., not occupied by the MEMS chip) can be filled with an acoustically transparent material, such as a gel, e.g., to acoustically coupe the tubing to the MEMs chip. Note that an acoustically transparent material refers to any material that allows sound to pass through the material without much barrier or resistance. Note further, that each of the ultrasonic transducer elements can be individually addressable to enable phased array focusing. In some instances, the MEMS chip can be micro-machined onto a substrate, e.g., such as a silicon substrate. In some instances, the array of ultrasonic transducers can be capacitive micro-machined ultrasonic transducers. In some instances, signals can be routed to the MEMS chip using a multi-layer flexible printed circuit board (flex-PCB) inserted into the interior of the tubing interior. In addition, wire-bonds can connect the flex-PCB to the MEMS chips. Signals can then be routed to the ultrasonic transducer elements using traces contained on the MEMS chip. Thus, probe 400 can introduce stimulation provided by LIFU while preserving traditional DBS functionality and the construction of probe 400 can maintain the mechanical flexibility of conventional DBS along all regions of the tube except the 1 cm span occupied by the MEMS chip. Note that in some instances, flexible MEMs can be introduced to maintain mechanical flexibility of the MEMs chip as well.

In some instances, the probe 400 can be configured for intra-acoustic medium ultrasonic modulation via insertion into an acoustic medium. In some instances, the ultrasonic modulation can be configured to affect behavior of neurons. In some instances, the ultrasonic modulation can be configured to alter behavior of a blood-brain barrier.

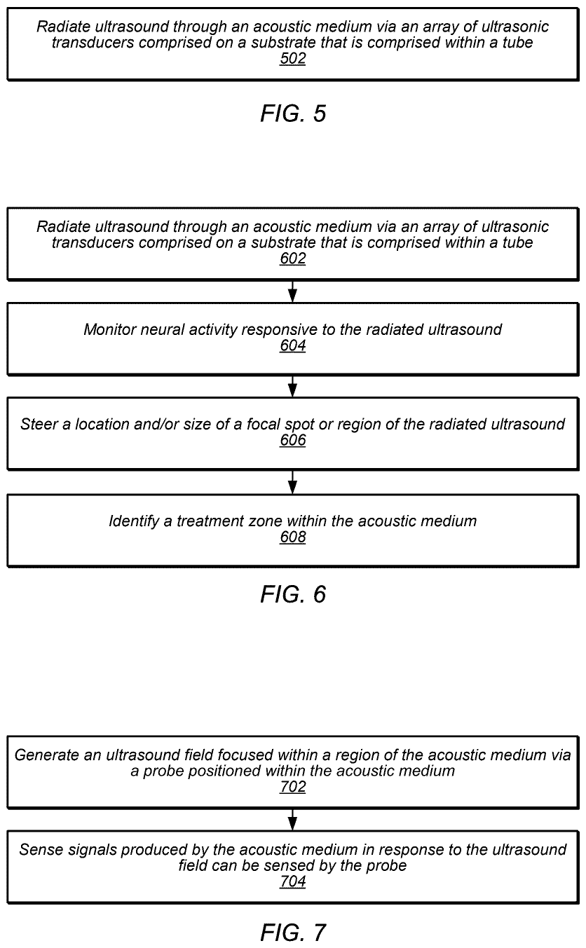

FIG. 5 illustrates a block diagram of an example of a method for intra-acoustic medium ultrasonic neuromodulation, according to some embodiments. The method shown in FIG. 5 can be used in conjunction with any of the systems, methods, or devices shown in the Figures, among other devices. In various embodiments, some of the method elements shown can be performed concurrently, in a different order than shown, or can be omitted. Additional method elements can also be performed as desired. As shown, this method can operate as follows.

At 502, ultrasound can be radiated through an acoustic medium, e.g., via an array of ultrasonic transducers positioned within the acoustic medium. In some instances, the array of ultrasonic transducers can be comprised on a substrate that resides in an interior of a tube to form an ultrasonic probe, e.g., such as probes 100 and/or 400 described herein. The tube can be a multi-lumen tube, at least in some instances. Additionally, a volume between the substrate and an interior wall of the tube can be filled with an acoustic substance (e.g., an acoustically transparent material, such as a gel) configured to provide acoustic coupling between the substrate and the tube. In some instances, the array of ultrasonic transducers can include (e.g., comprise) at least 10 (e.g., 10 or more) ultrasonic transducers. The ultrasonic transducers in the array of ultrasonic transducers can be spaced longitudinally and/or radially about the ultrasonic probe, which can be a cylindrical probe, e.g., a needle-style probe. Additionally, in some instances, each ultrasonic transducer in the array of ultrasonic transducers can be individually addressable. Further, in some instances, each ultrasonic transducer in the array of ultrasonic transducers can be independently controlled to produce the radiated ultrasound. In some instances, the substrate can be a MEMS chip. In some instances, an ultrasonic transducer (and/or an ultrasonic transducer elements) can be a capacitive micromachined ultrasonic transducer (CMUT), piezoelectric micromachined ultrasonic transducer (PMUT), and/or a 3-D printed piezoelectric element. In some instances, the array of ultrasonic transducers can include a 24-element array spanning a distance of approximately 1 centimeter along the interior of the tubing.

In some instances, a location and/or a size of a focal spot, point, and/or region of the radiated ultrasound can be based, at least in part, on input signals to each ultrasonic transducer in the array of ultrasonic transducers. In some instances, a location of the focal spot, point, and/or region can be dependent on a length, L, of the array of ultrasonic transducers. For example, the location of the focal spot, point, and/or region can be between 0.1L and 2L from the array of ultrasonic transducers. In some instances, a diameter of the focal spot, point, and/or region can be less than 150 micrometers. In some instances, a diameter of the focal spot, point, and/or region can be less than 100 micrometers. In some instances, a frequency of the ultrasound can be between 0.25 MHz to 10 MHz. In some instances, the focal spot, point, and/or region can include (e.g., comprise) a group of neurons.

In some instances, neural activity can be actuated responsive to the radiated ultrasound.

In some instances, neural activity can be sensed responsive to the radiated ultrasound, e.g., via one or more sensing electrodes positioned within the acoustic medium. The one or more sensing electrodes can be configured to sense local field potentials. Thus, in at least some instances, the one or more sensing electrodes can be one or more local field potential sensors.

In some instances, an electrical field in the acoustic medium can be generated, e.g., via one or more electrical transducers. In such instances, the neural activity actuated responsive to the radiated ultrasound can also include neural activity actuated responsive to the generated electrical field. In some instances, the one or more electrical transducers can be configured for electrical deep brain stimulation.

In some instances, a probe diameter (and/or diameter of the probe) can be approximately one twenty fourth of a probe length. Further, in some instances, local field potential sensors can also be included (e.g., comprised) on the probe. In some instances, the ultrasonic probe can be flexible. In other instances, the ultrasonic probe can be approximately and/or substantially rigid, e.g., at least in comparison to the acoustic medium. In some instances, the ultrasonic probe can be made, at least partially, of (e.g., comprised, at least partially, of) acoustically active material. In some instances, an ultrasonic transducer of the array of ultrasonic transducers can include an electrode pair. In other words, each ultrasonic transducer of the array of ultrasonic transducers can be and/or include an electrode pair. In some instances, the ultrasonic probe can include one or more electrical transducers.

In some instances, a location of the focal spot or region of the radiated ultrasound can be on or in immediate proximity of a surface of the probe thereby enabling substantially direct stimulation and excitation of a small group of neurons. In addition, the location can coincide with a location of at least one sensing electrode of the one or more sensing electrodes.

FIG. 6 illustrates a block diagram of another example of a method for intra-acoustic medium ultrasonic neuromodulation, according to some embodiments. The method shown in FIG. 6 can be used in conjunction with any of the systems, methods, or devices shown in the Figures, among other devices. In various embodiments, some of the method elements shown can be performed concurrently, in a different order than shown, or can be omitted. Additional method elements can also be performed as desired. As shown, this method can operate as follows.

At 602, ultrasound can be radiated through an acoustic medium, e.g., via an array of ultrasonic transducers positioned within the acoustic medium. In some instances, the array of ultrasonic transducers can be comprised on a substrate that resides in an interior of a tube to form an ultrasonic probe, e.g., such as probes 100 and/or 400 described herein. The tube can be a multi-lumen tube, at least in some instances. Additionally, a volume between the substrate and an interior wall of the tube can be filled with an acoustic substance (e.g., an acoustically transparent material, such as a gel) configured to provide acoustic coupling between the substrate and the tube. In some instances, the array of ultrasonic transducers can include (e.g., comprise) at least 10 (e.g., 10 or more) ultrasonic transducers. The ultrasonic transducers in the array of ultrasonic transducers can be spaced longitudinally and/or radially about the ultrasonic probe, which can be a cylindrical probe, e.g., a needle-style probe. Additionally, in some instances, each ultrasonic transducer in the array of ultrasonic transducers can be individually addressable. Further, in some instances, each ultrasonic transducer in the array of ultrasonic transducers can be independently controlled to produce the radiated ultrasound. In some instances, the substrate can be a MEMS chip. In some instances, an ultrasonic transducer (and/or an ultrasonic transducer elements) can be a capacitive micromachined ultrasonic transducer (CMUT), piezoelectric micromachined ultrasonic transducer (PMUT), and/or a 3-D printed piezoelectric element. In some instances, the array of ultrasonic transducers can include a 24-element array spanning a distance of approximately 1 centimeter along the interior of the tubing.

In some instances, a location and/or a size of a focal spot, point, and/or region of the radiated ultrasound can be based, at least in part, on input signals to each ultrasonic transducer in the array of ultrasonic transducers. In some instances, a location of the focal spot, point, and/or region can be dependent on a length, L, of the array of ultrasonic transducers. For example, the location of the focal spot, point, and/or region can be between 0.1L and 2L from the array of ultrasonic transducers. In some instances, a diameter of the focal spot, point, and/or region can be less than 150 micrometers. In some instances, a diameter of the focal spot, point, and/or region can be less than 100 micrometers. In some instances, a frequency of the ultrasound can be between 0.25 MHz to 10 MHz. In some instances, the focal spot, point, and/or region can include (e.g., comprise) a group of neurons.

At 604, neural activity can be monitored responsive to the radiated ultrasound. The neural activity can be associated with patient behavior and/or patient perception, at least in some instances. The neural activity can be monitored based on sensed local field potentials, at least in some instances. Thus, in at least some instances, local field potential sensors can be used to monitor neural activity.

At 606, a location and/or size of a focal spot or region of the radiated ultrasound can be steered, e.g., based on the monitoring.

At 608, a treatment zone, e.g., a group and/or volume of neurons, within the acoustic medium can be identified, e.g., based on the steering. In other words, as the location and/or size of the focal spot or region of the radiated ultrasound is steered through the acoustic medium, patient behavior/response can be monitored (e.g., via sensed neural activity and/or observation) to determine and/or identify the group and/or volume of neurons for treatment.

In some instances, an electrical field in the acoustic medium can be generated, e.g., via one or more electrical transducers. In such instances, the neural activity actuated responsive to the radiated ultrasound can also include neural activity actuated responsive to the generated electrical field. In some instances, the one or more electrical transducers can be configured for electrical deep brain stimulation.

In some instances, a probe diameter (and/or diameter of the probe) can be approximately one twenty fourth of a probe length. Further, in some instances, the local field potential sensors can also be included (e.g., comprised) on the probe. In some instances, the ultrasonic probe can be flexible. In other instances, the ultrasonic probe can be approximately and/or substantially rigid, e.g., at least in comparison to the acoustic medium. In some instances, the ultrasonic probe can be made, at least partially, of (e.g., comprised, at least partially, of) acoustically active material. In some instances, an ultrasonic transducer of the array of ultrasonic transducers can include an electrode pair. In other words, each ultrasonic transducer of the array of ultrasonic transducers can be and/or include an electrode pair. In some instances, the ultrasonic probe can include one or more electrical transducers.

In some instances, a location of the focal spot or region of the radiated ultrasound can be on or in immediate proximity of a surface of the probe thereby enabling substantially direct stimulation and excitation of a small group of neurons. In addition, the location can coincide with a location of at least one sensing electrode of the one or more sensing electrodes.

FIG. 7 illustrates a block diagram of a further example of a method for intra-acoustic medium ultrasonic neuromodulation, according to some embodiments. The method shown in FIG. 7 can be used in conjunction with any of the systems, methods, or devices shown in the Figures, among other devices. In various embodiments, some of the method elements shown can be performed concurrently, in a different order than shown, or can be omitted. Additional method elements can also be performed as desired. As shown, this method can operate as follows.

At 702, a probe can be positioned within an acoustic medium (e.g., such as a brain of a living subject).

At 702, an ultrasound field focused within a region of the acoustic medium can be generated via a probe positioned within the acoustic medium, e.g., via the array of ultrasonic transducer elements. As an example, when the acoustic medium is a brain of a living subject, the ultrasound field can modulate neural activity. In some instances, generation of the ultrasound field can include steering and/or shaping the ultrasound field using control signals applied to the array of ultrasonic transducer elements. In some instances, generation of the ultrasound field can include creating one or more focal regions that are movable relative to the probe. In some instances, the probe can include an array of ultrasonic transducer elements. In some instances, the array of ultrasonic transducer elements can be comprised on a substrate that resides in an interior of a tube to form the probe, e.g., such as probes 100 and/or 400 described herein. In some instances, the probe can be a flexible and/or semi-flexible structure configured to accommodate insertion into the acoustic medium using a pre-determined trajectory defined via imaging of the acoustic medium. In some instances, the array of ultrasonic transducer elements can be arranged along at least one of a longitudinal axis or a circumferential surface of the probe.

At 704, signals produced by the acoustic medium in response to the ultrasound field can be sensed by the probe, e.g., via one or more sensing elements on the probe. For example, when the acoustic medium is a brain of a living subject, the signals can be associated with neuronal activity modulated in response to the ultrasound field. In some instances, the signals can include local field potentials

In some instances, one or more ultrasound parameters can be adjusted. The one or more parameters can include at least one of intensity, frequency, phase, duty cycle, or focus location. In some cases, e.g., such as when the signals are associated with neuronal activity, adjustment of the one or more parameters can be based, at least in part, on neural response.

Embodiments of the present disclosure may be realized in any of various forms. For example, some embodiments may be realized as a computer-implemented method, a computer-readable memory medium (e.g., a non-transitory computer-readable memory medium), and/or a computer system. Other embodiments may be realized using one or more custom-designed hardware devices such as ASICs. Still other embodiments may be realized using one or more programmable hardware elements such as FPGAs.

In some embodiments, a non-transitory computer-readable memory medium may be configured so that it stores program instructions and/or data, where the program instructions, if and/or when executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a computer program, if and/or when executed by a computer system, may cause the computer system to perform a method, e.g., any of the method embodiments described herein, or, any combination of the method embodiments described herein, or, any subset of any of the method embodiments described herein, or, any combination of such subsets.

In some embodiments, a device may be configured to include a processor (or a set of processors) and a memory medium, where the memory medium stores program instructions or a computer program, where the processor is configured to read and execute the program instructions or computer program from the memory medium, where the program instructions are, or computer program is, executable to implement a method, e.g., any of the various method embodiments described herein (or, any combination of the method embodiments described herein, or, any subset of any of the method embodiments described herein, or, any combination of such subsets). The device may be realized in any of various forms.

Although the embodiments above have been described in considerable detail, numerous variations and modifications will become apparent to those skilled in the art once the above disclosure is fully appreciated. It is intended that the following claims be interpreted to embrace all such variations and modifications.

We claim:

1. An ultrasonic probe, comprising:
an array of ultrasonic transducers;
a substrate, wherein the array of ultrasonic transducers is comprised on the substrate; and
a tube, wherein the substrate is comprised within an interior of the tube; and
wherein the ultrasonic probe is configured for intra-acoustic medium ultrasonic modulation via insertion into an acoustic medium.

2. The ultrasonic probe of claim 1,
wherein a volume between the substrate and an interior wall of the tube is filled with an acoustic substance configured to provide acoustic coupling between the substrate and the tube.

3. The ultrasonic probe of claim 2,
wherein the acoustic substance comprises a gel.

4. The ultrasonic probe of claim 2,
wherein the acoustic substance is an acoustically transparent material.

5. The ultrasonic probe of claim 1,
wherein the ultrasonic probe is configured to:
radiate, via the array of ultrasonic transducers positioned within the acoustic medium, ultrasound through the acoustic medium, wherein one or more of a location or size of a focal spot or region of the radiated ultrasound is based, at least in part, on input signals to each ultrasonic transducer in the array of ultrasonic transducers.

6. The ultrasonic probe of claim 5,
wherein a location of the focal spot or region is dependent on a length, L, of the array of ultrasonic transducers, and wherein the location of the focal spot or region is between 0.1L and 2L from the array of ultrasonic transducers.

7. The ultrasonic probe of claim 5,
wherein the focal spot or region comprises a group of neurons.

8. The ultrasonic probe of claim 5,
wherein each ultrasonic transducer in the array of ultrasonic transducers is individually addressable; and
wherein each ultrasonic transducer in the array of ultrasonic transducers is independently controlled to produce the radiated ultrasound.

9. The ultrasonic probe of claim 1, further comprising:
one or more electrical transducers positioned on the tube and configured to generate an electrical field in the acoustic medium, wherein the one or more electrical transducers are configured to generate an electric field that affects behavior of neurons.

10. The ultrasonic probe of claim 1,
wherein the ultrasonic probe is flexible.

11. The ultrasonic probe of claim 1,
wherein the ultrasonic modulation is configured to affect behavior of neurons or alter behavior of a blood-brain barrier.

12. The ultrasonic probed of claim 1,
wherein the substrate comprises a silicon substrate.

13. The ultrasonic probed of claim 1,
wherein the array of ultrasonic transducers are capacitive micro-machined ultrasonic transducers.

14. A method for intra-acoustic medium ultrasonic modulation, comprising:
radiating, via an array of ultrasonic transducers arranged on an ultrasonic probe inserted into an acoustic medium, ultrasound through the acoustic medium, wherein the array of ultrasonic transducers is comprised on a substrate that resides within an interior of a tube to form the ultrasonic probe.

15. The method of claim 14, further comprising:
monitoring, neural activity responsive to the radiated ultrasound, wherein the neural activity is associated with patient behavior and/or patient perception.

16. The method of claim 15, further comprising:
steering, based on the monitoring, at least one of a location of a focal spot or region or a size of the focal spot or region of the radiated ultrasound; and
identifying, based on the steering, a treatment zone within the acoustic medium.

17. The method of claim 16,
wherein a location of the focal spot or region is dependent on a length, L, of the array of ultrasonic transducers, and wherein the location of the focal spot or region is between 0.1L and 2L from the array of ultrasonic transducers.

18. A non-transitory computer readable memory medium storing program instructions executable by at least one processor to perform operations comprising:
radiating, via an array of ultrasonic transducers arranged on an ultrasonic probe inserted into an acoustic medium, ultrasound through the acoustic medium, wherein the array of ultrasonic transducers is comprised on a substrate that resides within an interior of a tube to form the ultrasonic probe; and
steering, based on monitoring of neural activity responsive to the radiated ultrasound, at least one of a location of a focal spot or region or a size of the focal spot or region of the radiated ultrasound.

19. The non-transitory computer readable memory medium of claim 18,
wherein a volume between the substrate and an interior wall of the tube is filled with an acoustic substance configured to provide acoustic coupling between the substrate and the tube; and wherein the acoustic substance is an acoustically transparent material.

20. The non-transitory computer readable memory medium of claim 18,
wherein each ultrasonic transducer in the array of ultrasonic transducers is individually addressable; and
wherein each ultrasonic transducer in the array of ultrasonic transducers is independently controlled to produce the radiated ultrasound.
